SEQUENCE LISTING (15 sequences)

SEQ ID NO: 1 - protein, 182 aa, Bacillus cibi [sequence omitted]

SEQ ID NO: 2 - protein, 204 aa, Aspergillus oryzae
KTGSGDSQSDPIKADLEVKGQSALPFDVDCWAILCKGAPNVLQRVNEKTKNSNRDRSGANKGPFKDPQKWGIKALPPKNPSWSAQDFKSPEEYAFASSLQGGTNAILAPVNLASQNSQGGVLNGFYSANKVAQFDPSKPQQTKGTWFQITKFTGAAGPYCKALGSNDKSVCDKNKNIAGDWGFDPAKWAYQYDEKNNKFNYVGK

SEQ ID NO: 3 - protein, 5 aa, synthetic construct
GPYCK

SEQ ID NO: 4 - protein, 5 aa, synthetic construct
WFZIT

SEQ ID NO: 5 - DNA, 19 bp, synthetic construct
  source 1..19: mol_type = other DNA
tacgcggttaaagtcaatg

SEQ ID NO: 6 - DNA, 20 bp, synthetic construct
  source 1..20: mol_type = other DNA
acaaagcattctgaagctgg

SEQ ID NO: 7 - DNA, 19 bp, synthetic construct
  source 1..19: mol_type = other DNA
gaataaagaggaggatccg

SEQ ID NO: 8 - DNA, 20 bp, synthetic construct
  source 1..20: mol_type = other DNA
gggcggagcagacctcgaac

SEQ ID NO: 9 - DNA, 20 bp, synthetic construct
  source 1..20: mol_type = other DNA
cgacgatcatggaacggatt

SEQ ID NO: 10 - DNA, 24 bp, synthetic construct
  source 1..24: mol_type = other DNA
tcttcattctgatgaaggttgttc

SEQ ID NO: 11 - DNA, 21 bp, synthetic construct
  source 1..21: mol_type = other DNA
ttcgcaggatagccaattgta

SEQ ID NO: 12 - DNA, 24 bp, synthetic construct
  source 1..24: mol_type = other DNA
acagcggatcttcaggaaacacga

SEQ ID NO: 13 - DNA, 22 bp, synthetic construct
  source 1..22: mol_type = other DNA
cctggaatgctgttggattgta

SEQ ID NO: 14 - DNA, 20 bp, synthetic construct
  source 1..20: mol_type = other DNA
ctgtctcatggcagcatctt

SEQ ID NO: 15 - DNA, 24 bp, synthetic construct
  source 1..24: mol_type = other DNA
tgatttctctctcagctgaaacga

(19) United States
(12) Patent Application Publication
(10) Pub. No.: US 2025/0109373 A1
(43) Pub. Date: Apr. 3, 2025

(54) REDUCTION OF RESIDUAL DNA IN MICROBIAL FERMENTATION PRODUCTS

(71) Applicant: Novozymes A/S, Bagsvaerd (DK)

(72) Inventors: Carsten HJORT, Smoerum (DK); Risto CUKALEVSKI, Lund (SE); Joen SVINDT, Copenhagen (DK); Christian Lund CHRISTENSEN, Birkeroed (DK); Karsten Brandt JAKOBSEN, Tune, Greve (DK)

(73) Assignee: Novozymes A/S, Bagsvaerd (DK)

(21) Appl. No.: 18/723,211

(22) PCT Filed: Dec. 23, 2022
(86) PCT No.: PCT/EP2022/087724

§ 371 Date: Jun. 21, 2024

(30) Foreign Application Priority Data
Dec. 23, 2021 (EP) 21217519.4
Nov. 7, 2022 (EP) 22205881.0

(51) Int. Cl. C12N 1/08; C12N 9/22; C12P 21/02

(52) U.S. Cl. CPC C12N 1/08; C12N 9/22; C12P 21/02

(57) ABSTRACT

The invention provides a method for reducing the amount of DNA in a microbial fermentation product by adding a fungal DNase from Aspergillus oryzae to the resulting fermentation product during the recovery process.

### REFERENCE TO A SEQUENCE LISTING

This application contains a Sequence Listing in computer readable form. The computer readable form is incorporated herein by reference.

### FIELD OF THE INVENTION

The present invention relates to reduction of residual recombinant DNA in microbial fermentation products using a fungal DNase.

### BACKGROUND

Production of protein products by fermentation is a well-known process and it is used for production in industrial scale of many different proteins of interest. During fermentation some of the host cells producing the protein product of interest will break and the content of the cells, including DNA, will be released to the fermentation broth. Furthermore, in some fermentations the protein of interest is produced as an intracellular product. This means that the cells must be disrupted/lysed, for example by homogenization, before the recovery and purification process following the fermentation, and this inevitably results in the release of significant amounts of DNA into the fermentation broth that subsequently may end up in the final protein product as residual DNA.

It may be desirous to avoid residual DNA from the host cells producing the protein of interest, e.g., due to environmental or health concerns. This is a particular concern with recombinant DNA.

There is therefore a need for reducing residual DNA in fermentation products.

### SUMMARY OF THE INVENTION

The present invention provides, in a first aspect, a method for reducing the amount of DNA in a microbial fermentation product, comprising
(a) providing a fermentation broth comprising microbial host cells, recombinant DNA from the microbial host cells, and a protein of interest produced by the microbial host cells;
(b) subjecting the fermentation broth to a flocculation or precipitation step to provide a fermentation broth supernatant; and
(c) subjecting the fermentation broth supernatant to a membrane filtration step to provide a fermentation product, where the membrane has a size exclusion limit of less than 100 kDa or less than 1 μm;
wherein a fungal DNase or a variant thereof is added to the fermentation broth before, in, or after step (a), to the fermentation broth supernatant after step (b), or to the fermentation product after step (c).

In another aspect, the invention provides a microbial fermentation product comprising less than 10 ng/g, less than 5 ng/g, less than 1 ng/g, or less than 0.1 ng/g of recombinant DNA.

Other aspects and embodiments of the invention are apparent from the description and examples.

Unless otherwise indicated, or if it is apparent from the context that something else is meant, all percentages are percentage by weight (% w/w).

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the specification and relevant art and should not be interpreted in an idealized or overly formal sense unless expressly so defined herein. For the sake of brevity and/or clarity, well-known functions or constructions may not be described in detail.

As used herein, the singular forms “a,” “an,” and “the” are intended to include the plural forms as well, unless the context clearly indicates otherwise.

### Definitions

Sequence identity: The relatedness between two amino acid sequences or between two nucleotide sequences is described by the parameter “sequence identity”.

For purposes of the present invention, the sequence identity between two amino acid sequences is determined as the output of “longest identity” using the Needleman-Wunsch algorithm (Needleman and Wunsch, 1970, J. Mol. Biol. 48:443-453) as implemented in the Needle program of the EMBOSS package (EMBOSS: The European Molecular Biology Open Software Suite, Rice et al., 2000, Trends Genet. 16:276-277), preferably version 6.6.0 or later. The parameters used are a gap open penalty of 10, a gap extension penalty of 0.5, and the EBLOSUM62 (EMBOSS version of BLOSUM62) substitution matrix. In order for the Needle program to report the longest identity, the -nobrief option must be specified in the command line. The output of Needle labeled “longest identity” is calculated as follows:

(Identical Residues×100)/(Length of Alignment−Total Number of Gaps in Alignment)

Cell debris: The term “cell debris” refers to cell walls and other insoluble cellular components that are released after disruption of the cell wall/membrane, for example after disruption/lysis/homogenization of microbial cells. Even without intentionally disrupting the cell wall/membrane, some microbial cells may break in a fermentation process and release cell debris.

### Sequences

SEQ ID NO: 1: amino acid sequence of a DNase from Bacillus cibi.
SEQ ID NO: 2: amino acid sequence of a DNase from Aspergillus oryzae
SEQ ID NO: 3: amino acid sequence of a NUC3 DNase motif.
SEQ ID NO: 4: amino acid sequence of a NUC3 DNase motif.
SEQ ID NO: 5-15: nucleotide sequences of primers and probes used for dPCR (Example 1).

### DETAILED DESCRIPTION

We have found that fungal DNases, and in particular NUC3 nucleases, are highly efficient for reducing or removing residual DNA in fermentation products.

The present invention provides a method for reducing the amount of residual DNA in products comprising a protein of interest produced by fermentation of microbial host cells (in particular recombinant host cells) that express the protein of interest and secrete it into a fermentation broth, or accumulate it as an intracellular product. If the protein of interest is produced as an intracellular product, the microbial host cells may be homogenized before recovery and purification.

After fermentation and optional homogenization, the fermentation broth is subjected to a flocculation or precipitation step to provide a fermentation broth supernatant. This is effectively a solid/liquid separation that removes most of the (insoluble) host cells and cell debris, and retains an aqueous solution of the protein of interest and some soluble host cell components, such as residual host cell DNA.

Finally, the fermentation broth supernatant is filtered in a membrane filtration process to increase the purity of the protein of interest and provide a liquid fermentation product. The membrane filtration may remove higher and/or lower molecular weight components, depending on the type of membrane and filtration process. Some membrane filtration processes retain the filtrate (microfiltration), while others retain the retentate/permeate (ultrafiltration).

The method of the invention comprises adding a fungal DNase, or a variant thereof, to the fermentation medium before or during the fermentation, to the fermentation broth before flocculation/precipitation, to the fermentation broth supernatant before the membrane filtration, or to the fermentation product after the membrane filtration.

We have found that the fungal DNase can advantageously be applied to a fermentation product after a membrane filtration that reduces the amount of water and other low molecular compounds (ultra-filtration), and thus increases the concentration of the protein of interest and the residual host cell DNA.

When the fungal DNase or variant thereof is “added” or “applied”, according to the invention, this excludes (endogenous) production/expression of the DNase by the microbial host cells in the fermentation broth. The fungal DNase is an isolated or recovered enzyme which is added or applied from an external source. It is an advantage to apply the DNase from an external source because the production capacity of the microbial host cells is then used exclusively for producing the protein of (commercial) interest.

The DNA is considered removed when it is degraded to single nucleotides or oligonucleotides of less than 150 bp, for example as measured by digital PCR.

### Microbial Host Cell

The microbial host cell may be of any genus. The desired protein of interest may be homologous or heterologous to the host cell, which is capable of producing the protein of interest.

The term “homologous protein” or “native protein” means a protein encoded by a gene that is derived from the host cell in which it is produced.

The term “heterologous protein” means a protein encoded by a gene which is foreign to the host cell in which it is produced.

The term “recombinant host cell”, as used herein, means a host cell which harbors gene(s) encoding the desired protein and is capable of expressing said gene(s) to produce the desired protein. The desired protein coding gene(s) may be transformed, transfected, transduced, or the like, into the recombinant host cell using techniques well known in the art.

When the desired protein is a heterologous protein, the recombinant host cell capable of producing the desired protein is preferably of fungal or bacterial origin. The choice of recombinant host cell will to a large extent depend upon the gene coding for the desired protein and the source of said protein.

The term “wild-type host cell”, as used herein, refers to a host cell that natively harbors gene(s) coding for the desired protein and is capable of expressing said gene(s).

A mutant thereof may be a wild-type host cell in which one or more genes have been deleted, e.g., in order to enrich the desired protein preparation.

In a preferred embodiment, the recombinant or wild-type microbial host cell is a bacterium or a fungus.

The microbial host cell may be a yeast cell such as a Candida, Hansenula, Kluyveromyces, Pichia, Saccharomyces, Schizosaccharomyces, or Yarrowia strain. In another aspect, the strain is a Saccharomyces carlsbergensis, Saccharomyces cerevisiae, Saccharomyces diastaticus, Saccharomyces douglasii, Saccharomyces kluyveri, Saccharomyces norbensis, or Saccharomyces oviformis strain.

The microbial host cell may be a filamentous fungal strain such as an Acremonium, Agaricus, Alternaria, Aspergillus, Aureobasidium, Botryosphaeria, Ceriporiopsis, Chaetomidium, Chrysosporium, Claviceps, Cochliobolus, Coprinopsis, Coptotermes, Corynascus, Cryphonectria, Cryptococcus, Diplodia, Exidia, Filibasidium, Fusarium, Gibberella, Holomastigotoides, Humicola, Irpex, Lentinula, Leptospaeria, Magnaporthe, Melanocarpus, Meripilus, Mucor, Myceliophthora, Neocallimastix, Neurospora, Paecilomyces, Penicillium, Phanerochaete, Piromyces, Poitrasia, Pseudoplectania, Pseudotrichonympha, Rhizomucor, Schizophyllum, Scytalidium, Talaromyces, Thermoascus, Thielavia, Tolypocladium, Trichoderma, Trichophaea, Verticillium, Volvariella, or Xylaria strain.

In another aspect, the strain is an Acremonium cellulolyticus, Aspergillus aculeatus, Aspergillus awamori, Aspergillus foetidus, Aspergillus fumigatus, Aspergillus japonicus, Aspergillus nidulans, Aspergillus niger, Aspergillus oryzae, Chrysosporium inops, Chrysosporium keratinophilum, Chrysosporium lucknowense, Chrysosporium merdarium, Chrysosporium pannicola, Chrysosporium queenslandicum, Chrysosporium tropicum, Chrysosporium zonatum, Fusarium bactridioides, Fusarium cerealis, Fusarium crookwellense, Fusarium culmorum, Fusarium graminearum, Fusarium graminum, Fusarium heterosporum, Fusarium negundi, Fusarium oxysporum, Fusarium reticulatum, Fusarium roseum, Fusarium sambucinum, Fusarium sarcochroum, Fusarium sporotrichioides, Fusarium sulphureum, Fusarium torulosum, Fusarium trichothecioides, Fusarium venenatum, Humicola grisea, Humicola insolens, Humicola lanuginosa, Irpex lacteus, Mucor miehei, Myceliophthora thermophila, Neurospora crassa, Penicillium funiculosum, Penicillium purpurogenum, Phanerochaete chrysosporium, Thielavia achromatica, Thielavia albomyces, Thielavia albopilosa, Thielavia australeinsis, Thielavia fimeti, Thielavia microspora, Thielavia ovispora, Thielavia peruviana, Thielavia setosa, Thielavia spededonium, Thielavia subthermophila, Thielavia terrestris, Trichoderma harzianum, Trichoderma koningii, Trichoderma longibrachiatum, Trichoderma reesei, or Trichoderma viride strain.

In one aspect, the fungal host cell is a strain selected from the group consisting of Candida, Hansenula, Kluyveromyces, Pichia, Saccharomyces, Schizosaccharomyces, Yarrowia, Acremonium, Aspergillus, Fusarium, Humicola, Mucor, Myceliophthora, Neurospora, Penicillium, Thielavia, Tolypocladium, and Trichoderma.

In a more preferred embodiment, the filamentous fungal host cell is selected from the group consisting of Trichoderma and Aspergillus host cells, in particular a strain of Trichoderma harzianum, Trichoderma koningii, Trichoderma longibrachiatum, Trichoderma reesei, Trichoderma viridel, Aspergillus awamori, Aspergillus fumigatus, Aspergillus foetidus, Aspergillus japonicus, Aspergillus nidulans, Aspergillus niger or Aspergillus oryzae, especially a strain of Trichoderma reesei.

In another preferred embodiment, the recombinant or wild-type microbial host cell is a bacterium.

The recombinant host cell may comprise a single copy, or at least two copies, e.g., three, four, five, or more copies of the polynucleotide of the present invention.

The host cell may be any Gram-positive or Gram-negative bacterium. Gram-positive bacteria include, but are not limited to, Bacillus, Clostridium, Enterococcus, Geobacillus, Lactobacillus, Lactococcus, Oceanobacillus, Staphylococcus, Streptococcus, and Streptomyces. Gram-negative bacteria include, but are not limited to, Campylobacter, E. coli, Flavobacterium, Fusobacterium, Helicobacter, Ilyobacter, Neisseria, Pseudomonas, Salmonella, and Ureaplasma.

The host cell may be any Bacillus cell including, but not limited to, Bacillus alkalophilus, Bacillus amyloliquefaciens, Bacillus brevis, Bacillus circulans, Bacillus clausii, Bacillus coagulans, Bacillus firmus, Bacillus lautus, Bacillus lentus, Bacillus licheniformis, Bacillus megaterium, Bacillus pumilus, Bacillus stearothermophilus, Bacillus subtilis, and Bacillus thuringiensis cells. In an embodiment, the Bacillus cell is a Bacillus amyloliquefaciens, Bacillus licheniformis or Bacillus subtilis cell.

In one embodiment, the Bacillus cell is a Bacillus subtilis cell.

In another embodiment, the Bacillus cell is a Bacillus licheniformis cell.

For purposes of this invention, Bacillus classes/genera/species shall be defined as described in Patel and Gupta, 2020, Int. J. Syst. Evol. Microbiol. 70:406-438.

The bacterial host cell may also be any Streptococcus cell including, but not limited to, Streptococcus equisimilis, Streptococcus pyogenes, Streptococcus uberis, and Streptococcus equi subsp. Zooepidemicus cells.

The bacterial host cell may also be any Streptomyces cell including, but not limited to, Streptomyces achromogenes, Streptomyces avermitilis, Streptomyces coelicolor, Streptomyces griseus, and Streptomyces lividans cells.

Methods for introducing DNA into prokaryotic host cells are well-known in the art, and any suitable method can be used including but not limited to protoplast transformation, competent cell transformation, electroporation, conjugation, transduction, with DNA introduced as linearized or as circular polynucleotide. Persons skilled in the art will be readily able to identify a suitable method for introducing DNA into a given prokaryotic cell depending, e.g., on the genus. Methods for introducing DNA into prokaryotic host cells are for example described in Heinze et al., 2018, BMC Microbiology 18:56, Burke et al., 2001, Proc. Natl. Acad. Sci. USA 98:6289-6294, Choi et al., 2006, J. Microbiol. Methods 64:391-397, and Donald et al., 2013, J. Bacteriol. 195 (11): 2612-2620.

### Fungal DNase

The fungal DNase used in the invention is a deoxyribonucleases derived from a fungal microorganism, or a variant thereof. A DNase is any enzyme that catalyzes the hydrolytic cleavage of phosphodiester linkages in a DNA backbone, thus degrading DNA. There are two primary classifications based on the locus of activity. Exonucleases digest nucleic acids from the ends. Endonucleases act on regions in the middle of DNA molecules.

Examples of fungal DNases can be found, for example, in patent applications WO2017/059802 and WO2017/059801 (incorporated by reference), which disclose amino acid sequences encoding fungal DNases. Fungal DNases are wildtype DNases originating from fungal strains. A particularly preferred fungal DNase is the Aspergillus oryzae DNase shown as SEQ ID NO: 2.

The variant of the fungal DNase may have more than 60%, more than 70%, more than 80%, or more than 90% amino acid sequence identity to a fungal wildtype DNase.

In a particular embodiment, the fungal DNase or variant thereof has more than 60%, more than 70%, more than 80%, more than 90%, more than 95%, more than 96%, more than 97%, more than 98%, or more than 99% amino acid sequence identity to SEQ ID NO: 2. Alternatively, the fungal DNase or variant thereof has 1, 2, 3, 4, or 5 amino acid substitutions, deletions, or insertions, preferably 1, 2, 3, 4, or 5 conservative amino acid substitutions, as compared to SEQ ID NO: 2.

In a preferred embodiment, the fungal DNase or variant thereof is a NUC3 nuclease.

A subgroup of DNase_NucA_NucB (Pfam domain id PF14040, Pfam version 31.0 Finn (2016). Nucleic Acids Research, Database Issue 44: D279-D285) is termed NUC3. NUC3 nucleases contain the DNase_NucA_NucB domain and comprise the motif [LV][PTA][FY][DE][VAGPH]D[CFY][WY][AT][IM]L[CYQ] corresponding to amino acids at positions 24 to 35 in Aspergillus oryzae DNase with the amino acid sequence shown in SEQ ID NO: 2, and/or any of the motifs GPYCK (SEQ ID NO: 3) corresponding to amino acids at positions 157 to 161 in Aspergillus oryzae DNase with the amino acid sequence shown in SEQ ID NO: 2, or WF[QE]IT (SEQ ID NO: 4) corresponding to amino acids at positions 146 to 150 in Aspergillus oryzae DNase with the amino acid sequence shown in SEQ ID NO: 2.

Thus, the Aspergillus oryzae DNase shown as SEQ ID NO: 2 is a NUC3 DNase.

Amino acid changes in DNase variants, as described above, may be of a minor nature, that is conservative amino acid substitutions or insertions that do not significantly affect the folding and/or activity of the protein; small deletions, typically of 1-30 amino acids; small amino- or carboxyl-terminal extensions, such as an amino-terminal methionine residue; a small linker peptide of up to 20-25 residues; or a small extension that facilitates purification by changing net charge or another function, such as a poly-histidine tract, an antigenic epitope or a binding module.

Essential amino acids in a polypeptide (protein, enzyme) can be identified according to procedures known in the art, such as site-directed mutagenesis or alanine-scanning mutagenesis (Cunningham and Wells, 1989, Science 244:1081-1085). In the latter technique, single alanine mutations are introduced at every residue in the molecule, and the resultant molecules are tested for DNase activity to identify amino acid residues that are critical to the activity of the molecule. See also, Hilton et al., 1996, J. Biol. Chem. 271:4699-4708. The active site of the enzyme or other biological interaction can also be determined by physical analysis of structure, as determined by such techniques as nuclear magnetic resonance, crystallography, electron diffraction, or photoaffinity labeling, in conjunction with mutation of putative contact site amino acids. See, for example, de Vos et al., 1992, Science 255:306-312; Smith et al., 1992, J. Mol. Biol. 224:899-904; Wlodaver et al., 1992, FEBS Lett. 309:59-64. The identity of essential amino acids can also be inferred from an alignment with a related polypeptide, and/or be inferred from sequence homology and conserved catalytic machinery with a related polypeptide or within a polypeptide or protein family with polypeptides/proteins descending from a common ancestor, typically having similar three-dimensional structures, functions, and significant sequence similarity. Additionally or alternatively, protein structure prediction tools can be used for protein structure modelling to identify essential amino acids and/or active sites of polypeptides. See, for example, Jumper et al., 2021, “Highly accurate protein structure prediction with AlphaFold”, Nature 596:583-589.

Single or multiple amino acid substitutions, deletions, and/or insertions can be made and tested using known methods of mutagenesis, recombination, and/or shuffling, followed by a relevant screening procedure, such as those disclosed by Reidhaar-Olson and Sauer, 1988, Science 241:53-57; Bowie and Sauer, 1989, Proc. Natl. Acad. Sci. USA 86:2152-2156; WO 95/17413; or WO 95/22625. Other methods that can be used include error-prone PCR, CRISPR gene editing, phage display (e.g., Lowman et al., 1991, Biochemistry 30:10832-10837; U.S. Pat. No. 5,223,409; WO 92/06204), and region-directed mutagenesis (Derbyshire et al., 1986, Gene 46:145; Ner et al., 1988, DNA 7:127).

### Protein of Interest

The protein of interest is produced by the microbial host cell. Such proteins may be small (peptides; <50 amino acids) or large (polypeptides; >50 amino acids) biomolecules that perform a vast array of functions within living organisms, including catalyzing reactions, DNA replication, responding to stimuli, providing structure to cells and organisms, and transporting molecules from one location to another. Proteins are composed of chains of polymerized amino acids, which are folded in a very specific three-dimensional structure. The three-dimensional structure is critical for maintaining the function of the protein. Some chemicals can change the folding, or even unfold (denaturing) the three-dimensional structure, which will result in loss of function, such as loss of enzymatic activity.

In an embodiment, the proteins are polypeptides; preferably globular proteins/polypeptides. In another embodiment, the proteins are soluble at physiological conditions.

Proteins fall into at least four distinct groups, namely enzymes, cell signaling proteins, ligand binding proteins, and structural proteins.

In another embodiment, the at least one polypeptide of interest comprises a therapeutic polypeptide selected from the group consisting of an antibody, an antibody fragment, an antibody-based drug, a Fc fusion protein, an anticoagulant, a blood factor, a bone morphogenetic protein, an engineered protein scaffold, a growth factor, a blood clotting factor, a hormone, an interferon (such as an interferon alpha-2b), an interleukin, a lactoferrin, an alpha-lactalbumin, a beta-lactalbumin, an ovomucoid, an ovostatin, a cytokine, an obestatin, a human galactosidase (such as an human alpha-galactosidase A), a vaccine, a protein vaccine, and a thrombolytic.

Enzymes are described below.

Cell signaling and ligand binding proteins include proteins such as, for example, receptors, membrane proteins, ion channels, antibodies (e.g. single-domain antibodies), hormones, hemoglobin, and hemoglobin-like molecules. Heme-containing enzymes, such as peroxygenases and peroxidases, may comprise enzyme activity, or may be inactivated heme-containing enzyme variants.

Structural proteins provide stiffness and rigidity to otherwise-fluid biological components.

Preferably, the proteins are enzymes, cell signaling proteins, or ligand binding proteins; more preferably the proteins are enzymes.

### Enzymes

The protein of interest may be an enzyme (catalytic protein). When the protein is an enzyme, the amount of protein is active enzyme protein.

The term “active enzyme protein” is defined herein as the amount of catalytic protein(s), which exhibits enzymatic activity. This can be determined using an activity based analytical enzyme assay. In such assays, the enzyme typically catalyzes a reaction generating a colored compound. The amount of the colored compound can be measured and correlated to the concentration of the active enzyme protein. This technique is well-known in the art.

The enzyme(s) may be one or more enzymes, such as selected from the group consisting of hydrolases, lyases, transferases, proteases, amylases, glucoamylases, pectinases, pectate lyases, cellulases, xylanases, arabinases, arbinofuranosidases, mannanases, carrageenanases, xanthanases, endoglucanases, chitinases, asparaginases, lipases, phospholipases, cutinases, lysozymes, phytases, deamidases, transglutaminases, oxidoreductases (such as sugar oxidases, laccases, peroxidases, catalases), lactase, glucose isomerases, xylose isomerases, and esterases.

The enzyme may be a naturally occurring enzyme of bacterial or fungal origin, or it may be a variant derived from one or more naturally occurring enzymes by gene shuffling and/or by substituting, deleting or inserting one or more amino acids. Chemically modified or protein engineered mutants are included.

### Fermentation Broth

The present invention may be useful for any fermentation in industrial scale, e.g., for any fermentation having culture media of at least 50 liters, preferably at least 500 liters, more preferably at least 5,000 liters, even more preferably at least 50,000 liters.

The microorganism producing the protein of interest may be fermented by any method known in the art. The fermentation medium may be a minimal medium as described in, e.g., WO 98/37179, or the fermentation medium may be a complex medium comprising complex nitrogen and carbon sources, wherein the complex nitrogen source may be partially hydrolyzed as described in WO 2004/003216.

The fermentation may be performed as a batch, a repeated batch, a fed-batch, a repeated fed-batch or a continuous fermentation process.

In a fed-batch process, either none or part of the compounds comprising one or more nutrient(s) is added to the medium before the start of the fermentation and either all or the remaining part, respectively, of the compounds comprising one or more nutrients are fed during the fermentation process. The compounds which are selected for feeding can be fed together or separately to the fermentation process.

In a repeated fed-batch or a continuous fermentation process, the complete start medium is additionally fed during fermentation. The start medium can be fed together with or separately from the structural element feed(s). In a repeated fed-batch process, part of the fermentation broth comprising the biomass is removed at regular time intervals, whereas in a continuous process, the removal of part of the fermentation broth occurs continuously. The fermentation process is thereby replenished with a portion of fresh medium corresponding to the amount of withdrawn fermentation broth.

In a preferred embodiment of the invention, a fermentation broth from a fed-batch fermentation process is preferred.

In one embodiment, the fermentation broth is provided after a cultivation time of at least 24 hours, at least 48 hours, at least 72 hours, at least 96 hours, or at least 120 hours.

In a particular embodiment, the fermentation broth is provided after at least 120 hours of cultivation time.

According to the present invention, the fermentation broth may be diluted up to 2000% (w/w) with water; preferably the fermentation broth may be diluted 10-2000% (w/w) with water; more preferably the fermentation broth may be diluted 100-1500% (w/w) with water; more preferably the fermentation broth may be diluted 100-1000% (w/w) with water; more preferably the fermentation broth may be diluted 200-800% (w/w) with water.

Dilution with water means, according to the present invention, that the dilution medium may be water, or it may be an ultra-filtration permeate from the production of the protein of interest, or it may be a recycle of water from the production of the protein of interest, or it may be a condensate from a heater, or it may be any combination of the above mentioned, e.g., a mixture of water and an ultra-filtration permeate.

The fermentation broth comprises host cells (including, the host cells containing the gene encoding the polypeptide of interest which are used to produce the polypeptide of interest), cell debris, biomass, recombinant DNA from the bacterial host cells, fermentation media and/or fermentation products. In some embodiments, the composition is a cell-killed whole broth containing organic acid(s), killed cells and/or cell debris, and culture medium.

For example, fermentation broths are produced when microbial cultures are grown to saturation, incubated under carbon-limiting conditions to allow protein synthesis (e.g., expression of enzymes by host cells) and secretion into cell culture medium. The fermentation broth can contain unfractionated or fractionated contents of the fermentation materials derived at the end of the fermentation. Typically, the fermentation broth is unfractionated and comprises the spent culture medium and cell debris present after the microbial cells (e.g., Bacillus cells) are removed, e.g., by centrifugation. In some embodiments, the fermentation broth contains spent cell culture medium, extracellular enzymes, and viable and/or nonviable microbial cells.

The cell-killed whole broth or cell composition may contain the unfractionated contents of the fermentation materials derived at the end of the fermentation. Typically, the cell-killed whole broth or cell composition contains the spent culture medium and cell debris present after the microbial cells (e.g., Bacillus cells) are grown to saturation, incubated under carbon-limiting conditions to allow protein synthesis. In some embodiments, the cell-killed whole broth or cell composition contains the spent cell culture medium, recombinant DNA from the microbial host cells, extracellular enzymes, and killed microbial cells. In some embodiments, the microbial cells present in the cell-killed whole broth or composition can be permeabilized and/or lysed using methods known in the art.

A whole broth or cell composition as described herein is typically a liquid, but may contain insoluble components, such as killed cells, recombinant DNA from the microbial host cells, cell debris, culture media components, and/or insoluble enzyme(s). In some embodiments, insoluble components may be removed to provide a clarified liquid composition.

The whole broth formulations and cell compositions of the present invention may be produced by a method described in WO 90/15861 or WO 2010/096673.

### Flocculation/Precipitation

In order to flocculate the fermentation broth a divalent salt may be added to the fermentation broth, in particular a calcium salt and/or a magnesium salt, e.g., calcium chloride or magnesium chloride. A preferred embodiment is a calcium salt, in particular calcium chloride.

The salt may be added to the fermentation broth in a concentration of 0.01-10% (w/w) per kg fermentation broth (un-diluted); preferably 0.5-10% (w/w) per kg fermentation broth (undiluted); more preferably 1-9% (w/w) per kg fermentation broth (un-diluted); in particular 2-8% (w/w) per kg fermentation broth (un-diluted).

### Poly Aluminum Compound

To further improve removal of the DNA from the fermentation broth a poly aluminium compound may be added to the fermentation broth. Many Aluminium compounds are known to improve flocculation, e.g., Al₂(SO₄)₃, NaAlO₂, K₂Al₂O₄, AlCl₃, Al(NO₃)₃, Al-acetate, and Al-formate.

Particular useful poly aluminium chlorides include compounds of the formula Alₙ(OH)ₘCl₍₃ₙ₋ₘ₎ and poly aluminium chlorides and aluminium chlorohydrates with the CAS No.: 1327-41-9.

Examples of useful poly aluminium chlorides comprise aluminum chlorohydrate, GC850™ (Al₂(OH)₅Cl) obtainable from Gulbrandsen or NordPac 18 (available from Nordisk Aluminat A/S, Denmark) which is an aluminium complex with the brutto formula Al(OH)_{1,2}Cl_{1,8}. Another example of a useful poly aluminium chloride with the formula Al(OH)_{1,2}Cl_{1,8} is PAX-XL 100 (available from Kemira). Another two examples of useful poly aluminium chloride are PAC (available from Shanghai Haotian Water Treatment Equipment Co., Ltd supplied in solid form) or PAC (available from Tianjin Kairuite technology Ltd. Supplied in liquid form). Another example of useful poly aluminium chloride with the formula Al(OH)_{1,2}Cl_{1,8} is PAX18 (available from Kemira Water Solutions).

The concentration of the poly aluminium chloride will normally be in the range of 0.1-10% (w/w) calculated per kg fermentation broth (un-diluted); preferably in the range of 0.5-5% (w/w) calculated per kg fermentation broth (un-diluted).

After addition of the poly aluminium chloride, the pH may be adjusted. The pH may be adjusted to a pH within a range of pH 2 to pH 11. The pH may be adjusted with any acid or base as known in the art.

The poly aluminium chloride may also be added after the microorganism has been separated from the fermentation broth.

The poly aluminium chloride may also be added in two or more steps: e.g., before the microorganism is removed from the fermentation broth; and then again after the microorganism has been removed such as in the subsequent downstream process liquid.

### Polymers

Polymers may be used for particle aggregation. Anionic and cationic polymers are preferred. A useful cationic polymer may be a polyamine, and a useful anionic polymer may be a polyacrylamid. Useful polymer concentrations will normally be in the range of 0.5-20% (w/w) calculated per kg fermentation broth (un-diluted); preferably in the range of 1-10% (w/w) calculated per kg fermentation broth (un-diluted).

An example of a useful anionic polymer is Superfloc™ A 130 (Kemira). Examples of useful cationic polymers are Polycat™ (Kemira), C521 (Kemira), and C591 (Kemira).

### Filtration and Other Downstream Operations

The flocculated cell debris may be removed by methods known in the art such as, but not limited to, filtration, e.g., drum filtration, membrane filtration, filter-press dead end filtration, cross-flow filtration, or centrifugation.

The resulting fermentation supernatant may then be further processed or refined by methods known in the art. For example, the protein may be recovered by conventional procedures including, but not limited to, further filtration such as ultra-filtration and dia-filtration, extraction, spray-drying, evaporation, precipitation or crystallization.

The terms “recover” or “recovery” means the removal of a polypeptide from at least one fermentation broth component selected from the list of a cell, a nucleic acid, or other specified material, e.g., recovery of the polypeptide from the whole fermentation broth, or from the cell-free fermentation broth, by polypeptide crystal harvest, by filtration, e.g. depth filtration (by use of filter aids or packed filter medias, cloth filtration in chamber filters, rotary-drum filtration, drum filtration, rotary vacuum-drum filters, candle filters, horizontal leaf filters or similar, using sheed or pad filtration in framed or modular setups) or membrane filtration (using sheet filtration, module filtration, candle filtration, microfiltration, ultrafiltration in either cross flow, dynamic cross flow or dead end operation), or by centrifugation (using decanter centrifuges, disc stack centrifuges, hydro cyclones or similar), or by precipitating the polypeptide and using relevant solid-liquid separation methods to harvest the polypeptide from the broth media by use of classification separation by particle sizes. Recovery encompasses isolation and/or purification of the polypeptide.

The isolated protein may then be further purified and/or modified by a variety of procedures known in the art including, but not limited to, chromatography e.g. ion exchange, affinity, hydrophobic, chromatofocusing, and size exclusion and/or electrophoretic procedures e.g. preparative isoelectric focusing and/or differential solubility e.g., ammonium sulfate precipitation and/or extraction.

Microfiltration may involve a membrane having a size exclusion limit of more than 1000 kDa, more than 500 kDa, more than 100 kDa, or more than 50 kDa; and/or more than 5 μm, more than 1 μm, more than 0.5 μm, more than 0.4 μm, more than 0.3 μm, more than 0.2 μm, or more than 0.1 μm; or other filters with equivalent molecular weight exclusion properties; wherein the fermentation product is a filtrate of the microfiltration.

Subsequent to the microfiltration, or in a combined process, the fermentation product may be subjected to an ultra-filtration step, which involves a membrane having a size exclusion limit of more than 100 kDa, more than 80 kDa, more than 60 kDa, more than 50 kDa, more than 40 kDa, more than 30 kDa, more than 20 kDa, more than 15 kDa, more than 10 kDa, more than 5 kDa, or more than 1 kDa; or another filter with equivalent molecular weight exclusion properties.

The microbial fermentation product may be a liquid or solid formulation, or it may be used to prepare such formulations.

Liquid formulations may comprise polyols (polyhydric alcohols), for example in amounts of at least 10% w/w, at least 25% w/w, or at least 50% w/w. Polyols are alcohols with two or more hydroxyl groups. Useful polyols typically have a molecular weight lower than 500 g/mol.

Polyols include non-sugar polyols, such as glycerol, ethylene glycol, diethylene glycol, triethylene glycol, propylene glycol, dipropylene glycol, tripropylene glycol, polyethylene glycol (PEG), and sugar alcohols such as sorbitol, mannitol, erythritol, dulcitol, inositol, xylitol and adonitol. Polyols also include sugar polyols, such as mono- and disaccharides, like glucose, fructose, galactose, sucrose, lactose, maltose, and trehalose.

Solid formulations may be granulates prepared by for example high-shear granulation or fluid-bed granulation or combinations. Coatings with salt(s) or polymer(s) may also be applied.

### Detection of Residual Host Cell DNA

The term residual host cell DNA is including genomic DNA from the production strain and the fragment of DNA encoding for the protein of interest.

The detection and quantification of minute amounts of residual host cell DNA may be accomplished by various methods known in the art. Many methods are developed to measure specific single target sequences. Examples of methods are:
(a) a hybridization-based method for the detection of the specific DNA of defined origin with dot blots and hybridization of radioisotope-labeled DNA probes using random hexamers to generate representative probes covering the whole genome of the host cells;
(b) a quantitative PCR-based method for the detection of specific DNA of defined origin targeting a specific gene sequence for amplification and calibration using purified, species-matched, genomic DNA; and
(c) a qualitative PCR method for the detection of specific DNA of defined origin targeting a specific gene sequence for amplification and calibration using purified, species-matched, genomic DNA. A threshold is determined based on the lowest amount of genome DNA that can be detected using the method.

According to the present invention purification of trace DNA was accomplished by use of the FastDNA™ Spin Kit (MP Biomedicals). The eluted sample was then subjected to a PCR reaction using specific primers for a chromosomal locus on the host cell. Positive controls are included where known amounts of host DNA are added to PCR reactions in different concentrations. After the PCR reaction the samples are subjected to gel-electrophoresis and intensity of the DNA bands compared to estimate the concentration of the host DNA in the original sample. Detailed protocols for PCR are provided in Innis et al (1990) PCR Protocols, A Guide to methods and applications, Academic Press inc., N.Y.

Treatment of a fermentation broth or other protein preparation using the present method results in significant reduction in amounts of DNA being present in the fermentation broth, and preferably the DNA content is reduced to an undetectable level. A level is considered undetectable if PCR amplification of any segment of genomic DNA present in a single copy in a haploid genome followed by ethidium bromide staining gives no visible band.

Preferably the DNA level in the fermentation broth is reduced to a level below 1 μg/ml, preferably below 500 ng/ml, preferably below 200 ng/ml, preferably below 100 ng/ml, preferably below 50 ng/ml, preferably below 20 ng/ml, preferably below 10 ng/ml, preferably below 5 ng/ml, preferably below 2 ng/ml preferably below 1 ng/ml, and most preferred below 500 μg/ml.

In an embodiment, the microbial fermentation product comprises less than 10 ng/g, less than 5 ng/g, less than 1 ng/g, less than 0.5 ng/g, less than 0.1 ng/g, less than 0.05 ng/g, or less than 0.01 ng/g of recombinant DNA.

In an example of a typical regulatory environment, no detectable DNA, may be ascertained using a PCR-based assay with a detection limit of, for example 1, 5, 10 or 20 ng/ml enzyme preparation.

In addition, the use of the instant method in combination with conventional methods of removing DNA from fermentation broths or other protein preparation is also contemplated.

Some further embodiments of the invention include:

Embodiment 1. A method for reducing the amount of DNA in a microbial fermentation product, comprising
(a) providing a fermentation broth comprising microbial host cells, recombinant DNA from the microbial host cells, and a protein of interest produced by the microbial host cells;
(b) subjecting the fermentation broth to a flocculation or precipitation step to provide a fermentation broth supernatant; and
(c) subjecting the fermentation broth supernatant to a membrane filtration step to provide a fermentation product, where the membrane has a size exclusion limit of less than 100 kDa or less than 1 μm;
wherein a fungal DNase or a variant thereof is added to the fermentation medium before or during the fermentation, to the fermentation broth in or after step (a), to the fermentation broth supernatant after step (b), or to the fermentation product after step (c).

Embodiment 2. The method of embodiment 1, wherein the fungal DNase or variant thereof is added to the fermentation medium before or during the fermentation.

Embodiment 3. The method of embodiment 1, wherein the fungal DNase or variant thereof is added to the fermentation broth in step (a).

Embodiment 4. The method of embodiment 1, wherein the fungal DNase or variant thereof is added to the fermentation broth after step (a).

Embodiment 5. The method of embodiment 1, wherein the fungal DNase or variant thereof is added to the fermentation broth after step (b).

Embodiment 6. The method of embodiment 1, wherein the fungal DNase or variant thereof is added to the fermentation broth after step (c).

Embodiment 7. The method of any of the preceding embodiments, wherein the protein of interest is heterologous to the microbial host cells.

Embodiment 8. The method of any of the preceding embodiments, wherein the protein of interest is secreted into the fermentation broth by the microbial host cells.

Embodiment 9. The method of any of the preceding embodiments, wherein the protein of interest is not secreted into the fermentation broth by the microbial host cells.

Embodiment 10. The method of any of the preceding embodiments, wherein the fermentation broth in step (a) comprises the protein of interest in an amount of at least 0.1% w/w.

Embodiment 11. The method of any of the preceding embodiments, wherein the fermentation broth in step (a) comprises the protein of interest in an amount of at least 0.5% w/w.

Embodiment 12. The method of any of the preceding embodiments, wherein the fermentation broth in step (a) comprises the protein of interest in an amount of at least 1% w/w.

Embodiment 13. The method of any of the preceding embodiments, wherein the protein of interest is an enzyme, heme-containing protein, cell signaling protein, or ligand binding protein.

Embodiment 14. The method of any of the preceding embodiments, wherein the protein of interest is an enzyme.

Embodiment 15. The method of any of the preceding embodiments, wherein the membrane filtration in step (c) comprises a microfiltration step.

Embodiment 16. The method of the preceding embodiment, wherein the microfiltration membrane has a size exclusion limit of 0.1 μm to 10 μm.

Embodiment 17. The method of the preceding embodiment, wherein the microfiltration membrane has a size exclusion limit of 0.5 μm to 5 μm.

Embodiment 18. The method of any of the preceding embodiments, wherein the membrane filtration in step (c) comprises an ultra-filtration step.

Embodiment 19. The method of the preceding embodiment, wherein the ultra-filtration membrane has a size exclusion limit of less than 100 kDa.

Embodiment 20. The method of the preceding embodiment, wherein the ultra-filtration membrane has a size exclusion limit of less than 50 kDa.

Embodiment 21. The method of the preceding embodiment, wherein the ultra-filtration membrane has a size exclusion limit of less than 40 kDa.

Embodiment 22. The method of the preceding embodiment, wherein the ultra-filtration membrane has a size exclusion limit of less than 30 kDa.

Embodiment 23. The method of the preceding embodiment, wherein the ultra-filtration membrane has a size exclusion limit of less than 20 kDa.

Embodiment 24. The method of the preceding embodiment, wherein the ultra-filtration membrane has a size exclusion limit of less than 10 kDa.

Embodiment 25. The method of any of the preceding embodiments, wherein some or all of the microbial host cells in step (a) have been disrupted/homogenized mechanically or enzymatically.

Embodiment 26. The method of any of the preceding embodiments, wherein the fungal DNase or variant thereof is a wildtype fungal DNase or a variant thereof having at least 60% amino acid sequence identity to the wildtype fungal DNase, and where the variant exhibits DNase activity.

Embodiment 27. The method of embodiment 25, wherein the variant has at least 70% amino acid sequence identity to the wildtype fungal DNase.

Embodiment 28. The method of embodiment 25, wherein the variant has at least 80% amino acid sequence identity to the wildtype fungal DNase.

Embodiment 29. The method of embodiment 25, wherein the variant has at least 90% amino acid sequence identity to the wildtype fungal DNase.

Embodiment 30. The method of embodiment 25, wherein the variant has at least 95% amino acid sequence identity to the wildtype fungal DNase.

Embodiment 31. The method of embodiment 25, wherein the variant has at least 96% amino acid sequence identity to the wildtype fungal DNase.

Embodiment 32. The method of embodiment 25, wherein the variant has at least 97% amino acid sequence identity to the wildtype fungal DNase.

Embodiment 33. The method of embodiment 25, wherein the variant has at least 98% amino acid sequence identity to the wildtype fungal DNase.

Embodiment 34. The method of embodiment 25, wherein the variant has at least 99% amino acid sequence identity to the wildtype fungal DNase.

Embodiment 35. The method of any of the preceding embodiments, wherein the fungal DNase or variant thereof is a wildtype fungal DNase, or a variant thereof having 1, 2, 3, 4, or 5 substitutions, deletions, or insertions; preferably 1, 2, 3, 4, or 5 conservative substitutions.

Embodiment 36. The method of any of the preceding embodiments, wherein the fungal DNase or variant thereof is a wildtype fungal DNase, or a variant thereof having 1, 2, 3, 4, or 5 conservative substitutions.

Embodiment 37. The method of any of embodiments 25-35, wherein the wildtype fungal DNase has the amino acid sequence shown as SEQ ID NO: 2.

Embodiment 38. The method of any of the preceding embodiments, wherein the fungal DNase or variant thereof is a NUC3 nuclease.

Embodiment 39. The method of any of the preceding embodiments, wherein the fungal DNase or variant thereof comprises the DNase_NucA_NucB domain, and further comprises the motif [LV][PTA][FY][DE][VAGPH]D[CFY][WY][AT][IM]L[CYQ] and/or any of the motifs GPYCK (SEQ ID NO: 3) or WF[QE]IT (SEQ ID NO: 4).

Embodiment 40. The method of any of the preceding embodiments, wherein the fungal DNase or variant thereof comprises or consists of the amino acid sequence of SEQ ID NO: 2. Embodiment 41. The method of any of the preceding embodiments, wherein the microbial host cell is a bacterial host cell, a fungal host cell, or a yeast host cell; preferably, the microbial host cell is a bacterial host cell.

Embodiment 42. The method of any of the preceding embodiments, wherein the microbial host cell is a strain selected from the group consisting of Candida, Hansenula, Kluyveromyces, Pichia, Saccharomyces, Schizosaccharomyces, Yarrowia, Acremonium, Aspergillus, Fusarium, Humicola, Mucor, Myceliophthora, Neurospora, Penicillium, Thielavia, Tolypocladium, and Trichoderma.

Embodiment 43. The method of any of the preceding embodiments, wherein the microbial host cell is a strain of Pichia.

Embodiment 44. The method of any of the preceding embodiments, wherein the microbial host cell is a strain of Pichia pastoris.

Embodiment 45. The method of any of the preceding embodiments, wherein the microbial host cell is a strain selected from the group consisting of Bacillus, Streptomyces, Escherichia, Buttiauxella and Pseudomonas.

Embodiment 46. The method of any of the preceding embodiments, wherein the microbial host cell is a strain of Bacillus or Escherichia.

Embodiment 47. The method of any of the preceding embodiments, wherein the microbial host cell is a Bacillus host cell; preferably a Bacillus amyloliquefaciens, Bacillus licheniformis, or Bacillus subtilis host cell.

Embodiment 48. The method of any of the preceding embodiments, wherein the microbial fermentation product comprises less than 10 ng/g of recombinant DNA.

Embodiment 49. The method of any of the preceding embodiments, wherein the microbial fermentation product comprises less than 5 ng/g of recombinant DNA.

Embodiment 50. The method of any of the preceding embodiments, wherein the microbial fermentation product comprises less than 1 ng/g of recombinant DNA.

Embodiment 51. The method of any of the preceding embodiments, wherein the microbial fermentation product comprises less than 0.5 ng/g of recombinant DNA.

Embodiment 52. The method of any of the preceding embodiments, wherein the microbial fermentation product comprises less than 0.1 ng/g of recombinant DNA.

Embodiment 53. The method of any of the preceding embodiments, wherein the microbial fermentation product comprises less than 0.05 ng/g of recombinant DNA.

Embodiment 54. The method of any of the preceding embodiments, wherein the microbial fermentation product comprises less than 0.01 ng/g of recombinant DNA.

Embodiment 55. The method of any of the preceding embodiments, wherein the microbial fermentation product comprises the fungal DNase or variant thereof.

Embodiment 56. A method for reducing the amount of DNA in a microbial fermentation product, comprising
(a) providing a fermentation broth comprising microbial host cells, recombinant DNA from the microbial host cells, and a protein of interest produced by the microbial host cells; (b) subjecting the fermentation broth to a flocculation or precipitation step to provide a fermentation broth supernatant; and
(c) subjecting the fermentation broth supernatant to a membrane filtration step to provide a fermentation product, where the membrane has a size exclusion limit of less than 100 kDa or less than 1 μm;
wherein a DNase is added to the fermentation medium before or during the fermentation, to the fermentation broth in or after step (a), to the fermentation broth supernatant after step (b), or to the fermentation product after step (c);
wherein the DNase has at least 80% amino acid sequence identity to SEQ ID NO: 2; and
wherein the microbial fermentation product comprises less than 10 ng/g of recombinant DNA.

Embodiment 57. The method of embodiment 56, wherein the DNase is added to the fermentation medium before or during the fermentation.

Embodiment 58. The method of embodiment 56, wherein the DNase is added to the fermentation broth in step (a).

Embodiment 59. The method of embodiment 56, wherein the DNase is added to the fermentation broth after step (a).

Embodiment 60. The method of embodiment 56, wherein the DNase is added to the fermentation broth after step (b).

Embodiment 61. The method of embodiment 56, wherein the DNase is added to the fermentation broth after step (c).

Embodiment 62. The method of any of the preceding embodiments, wherein the protein of interest is heterologous to the microbial host cells.

Embodiment 63. The method of any of the preceding embodiments, wherein the protein of interest is secreted into the fermentation broth by the microbial host cells.

Embodiment 64. The method of any of the preceding embodiments, wherein the protein of interest is not secreted into the fermentation broth by the microbial host cells.

Embodiment 65. The method of any of the preceding embodiments, wherein the fermentation broth in step (a) comprises the protein of interest in an amount of at least 0.1% w/w.

Embodiment 66. The method of any of the preceding embodiments, wherein the fermentation broth in step (a) comprises the protein of interest in an amount of at least 0.5% w/w.

Embodiment 67. The method of any of the preceding embodiments, wherein the fermentation broth in step (a) comprises the protein of interest in an amount of at least 1% w/w.

Embodiment 68. The method of any of the preceding embodiments, wherein the protein of interest is an enzyme, heme-containing protein, cell signaling protein, or ligand binding protein.

Embodiment 69. The method of any of the preceding embodiments, wherein the protein of interest is an enzyme.

Embodiment 70. The method of the preceding embodiment, wherein the membrane has a size exclusion limit of less than 50 kDa.

Embodiment 71. The method of the preceding embodiment, wherein the membrane has a size exclusion limit of less than 40 kDa.

Embodiment 72. The method of the preceding embodiment, wherein the membrane has a size exclusion limit of less than 30 kDa.

Embodiment 73. The method of the preceding embodiment, wherein the membrane has a size exclusion limit of less than 20 kDa.

Embodiment 74. The method of the preceding embodiment, wherein the membrane has a size exclusion limit of less than 10 kDa.

Embodiment 75. The method of any of the preceding embodiments, wherein some or all of the microbial host cells in step (a) have been disrupted/homogenized mechanically or enzymatically.

Embodiment 76. The method of any of the preceding embodiments, wherein the DNase has at least 85% amino acid sequence identity to SEQ ID NO: 2.

Embodiment 77. The method of any of the preceding embodiments, wherein the DNase has at least 90% amino acid sequence identity to SEQ ID NO: 2.

Embodiment 78. The method of any of the preceding embodiments, wherein the DNase has at least 95% amino acid sequence identity to SEQ ID NO: 2.

Embodiment 79. The method of any of the preceding embodiments, wherein the DNase has at least 96% amino acid sequence identity to SEQ ID NO: 2.

Embodiment 80. The method of any of the preceding embodiments, wherein the DNase has at least 97% amino acid sequence identity to SEQ ID NO: 2.

Embodiment 81. The method of any of the preceding embodiments, wherein the DNase has at least 98% amino acid sequence identity to SEQ ID NO: 2.

Embodiment 82. The method of any of the preceding embodiments, wherein the DNase has at least 99% amino acid sequence identity to SEQ ID NO: 2.

Embodiment 83. The method of any of the preceding embodiments, wherein the DNase has 1, 2, 3, 4, or 5 substitutions, deletions, or insertions; preferably 1, 2, 3, 4, or 5 conservative substitutions as compared to the amino acid sequence shown as SEQ ID NO: 2.

Embodiment 84. The method of any of the preceding embodiments, wherein the DNase is a NUC3 nuclease.

Embodiment 85. The method of any of the preceding embodiments, wherein the DNase comprises the DNase_NucA_NucB domain, and further comprises the motif [LV][PTA][FY][DE][VAGPH]D[CFY][WY][AT][IM]L[CYQ] and/or any of the motifs GPYCK (SEQ ID NO: 3) or WF[QE]IT (SEQ ID NO: 4).

Embodiment 86. The method of any of the preceding embodiments, wherein the DNase has the amino acid sequence shown as SEQ ID NO: 2.

Embodiment 87. The method of any of the preceding embodiments, wherein the microbial host cell is a bacterial host cell, a fungal host cell, or a yeast host cell.

Embodiment 88. The method of any of the preceding embodiments, wherein the microbial host cell is a strain selected from the group consisting of Candida, Hansenula, Kluyveromyces, Pichia, Saccharomyces, Schizosaccharomyces, Yarrowia, Acremonium, Aspergillus, Fusarium, Humicola, Mucor, Myceliophthora, Neurospora, Penicillium, Thielavia, Tolypocladium, and Trichoderma.

Embodiment 89. The method of any of the preceding embodiments, wherein the microbial host cell is a strain of Pichia.

Embodiment 90. The method of any of the preceding embodiments, wherein the microbial host cell is a strain of Pichia pastoris.

Embodiment 91. The method of any of the preceding embodiments, wherein the microbial host cell is a bacterial host cell.

Embodiment 92. The method of any of the preceding embodiments, wherein the microbial host cell is a strain selected from the group consisting of Bacillus, Streptomyces, Escherichia, Buttiauxella and Pseudomonas.

Embodiment 93. The method of any of the preceding embodiments, wherein the microbial host cell is a strain of Bacillus or Escherichia.

Embodiment 94. The method of any of the preceding embodiments, wherein the microbial host cell is a Bacillus host cell; preferably a Bacillus amyloliquefaciens, Bacillus licheniformis, or Bacillus subtilis host cell.

Embodiment 95. The method of any of the preceding embodiments, wherein the microbial fermentation product comprises less than 5 ng/g of recombinant DNA.

Embodiment 96. The method of any of the preceding embodiments, wherein the microbial fermentation product comprises less than 1 ng/g of recombinant DNA.

Embodiment 97. The method of any of the preceding embodiments, wherein the microbial fermentation product comprises less than 0.5 ng/g of recombinant DNA.

Embodiment 98. The method of any of the preceding embodiments, wherein the microbial fermentation product comprises less than 0.1 ng/g of recombinant DNA.

Embodiment 99. The method of any of the preceding embodiments, wherein the microbial fermentation product comprises less than 0.05 ng/g of recombinant DNA.

Embodiment 100. The method of any of the preceding embodiments, wherein the microbial fermentation product comprises less than 0.01 ng/g of recombinant DNA.

Embodiment 101. The method of any of the preceding embodiments, wherein the microbial fermentation product comprises the DNase.

Embodiment 102. A method for reducing the amount of DNA in a microbial fermentation product, comprising
(a) providing a fermentation broth comprising Bacillus host cells, recombinant DNA from the Bacillus host cells, and an enzyme produced by the Bacillus host cells;
(b) subjecting the fermentation broth to a flocculation or precipitation step to provide a fermentation broth supernatant; and
(c) subjecting the fermentation broth supernatant to a membrane filtration step to provide a fermentation product, where the membrane has a size exclusion limit of less than 100 kDa or less than 1 μm;
wherein a DNase is added to the fermentation medium before or during the fermentation, to the fermentation broth in or after step (a), to the fermentation broth supernatant after step (b), or to the fermentation product after step (c);
wherein the DNase has at least 80% amino acid sequence identity to SEQ ID NO: 2; and
wherein the microbial fermentation product comprises less than 10 ng/g of recombinant DNA.

Embodiment 103. The method of embodiment 102, wherein the DNase is added to the fermentation medium before or during the fermentation.

Embodiment 104. The method of embodiment 102, wherein the DNase is added to the fermentation broth in step (a).

Embodiment 105. The method of embodiment 102, wherein the DNase is added to the fermentation broth after step (a).

Embodiment 106. The method of embodiment 102, wherein the DNase is added to the fermentation broth after step (b).

Embodiment 107. The method of embodiment 102, wherein the DNase is added to the fermentation broth after step (c).

Embodiment 108. The method of any of the preceding embodiments, wherein the enzyme is heterologous to the Bacillus host cells.

Embodiment 109. The method of any of the preceding embodiments, wherein the enzyme is secreted into the fermentation broth by the Bacillus host cells.

Embodiment 110. The method of any of the preceding embodiments, wherein the fermentation broth in step (a) comprises the enzyme in an amount of at least 0.1% w/w.

Embodiment 111. The method of any of the preceding embodiments, wherein the fermentation broth in step (a) comprises the enzyme in an amount of at least 0.5% w/w.

Embodiment 112. The method of any of the preceding embodiments, wherein the fermentation broth in step (a) comprises the enzyme in an amount of at least 1% w/w.

Embodiment 113. The method of the preceding embodiment, wherein the membrane has a size exclusion limit of less than 50 kDa.

Embodiment 114. The method of the preceding embodiment, wherein the membrane has a size exclusion limit of less than 40 kDa.

Embodiment 115. The method of the preceding embodiment, wherein the membrane has a size exclusion limit of less than 30 kDa.

Embodiment 116. The method of the preceding embodiment, wherein the membrane has a size exclusion limit of less than 20 kDa.

Embodiment 117. The method of the preceding embodiment, wherein the membrane has a size exclusion limit of less than 10 kDa.

Embodiment 118. The method of any of the preceding embodiments, wherein the DNase has at least 85% amino acid sequence identity to SEQ ID NO: 2.

Embodiment 119. The method of any of the preceding embodiments, wherein the DNase has at least 90% amino acid sequence identity to SEQ ID NO: 2.

Embodiment 120. The method of any of the preceding embodiments, wherein the DNase has at least 95% amino acid sequence identity to SEQ ID NO: 2.

Embodiment 121. The method of any of the preceding embodiments, wherein the DNase has at least 96% amino acid sequence identity to SEQ ID NO: 2.

Embodiment 122. The method of any of the preceding embodiments, wherein the DNase has at least 97% amino acid sequence identity to SEQ ID NO: 2.

Embodiment 123. The method of any of the preceding embodiments, wherein the DNase has at least 98% amino acid sequence identity to SEQ ID NO: 2.

Embodiment 124. The method of any of the preceding embodiments, wherein the DNase has at least 99% amino acid sequence identity to SEQ ID NO: 2.

Embodiment 125. The method of any of the preceding embodiments, wherein the DNase has 1, 2, 3, 4, or 5 substitutions, deletions, or insertions; preferably 1, 2, 3, 4, or 5 conservative substitutions as compared to the amino acid sequence shown as SEQ ID NO: 2.

Embodiment 126. The method of any of the preceding embodiments, wherein the DNase is a NUC3 nuclease.

Embodiment 127. The method of any of the preceding embodiments, wherein the DNase comprises the DNase_NucA_NucB domain, and further comprises the motif [LV][PTA][FY][DE][VAGPH]D[CFY][WY][AT][IM]L[CYQ] and/or any of the motifs GPYCK (SEQ ID NO: 3) or WF[QE]IT (SEQ ID NO: 4).

Embodiment 128. The method of any of the preceding embodiments, wherein the DNase has the amino acid sequence shown as SEQ ID NO: 2.

Embodiment 129. The method of any of the preceding embodiments, wherein the Bacillus host cell is a Bacillus amyloliquefaciens, Bacillus licheniformis, or Bacillus subtilis host cell.

Embodiment 130. The method of any of the preceding embodiments, wherein the microbial fermentation product comprises less than 5 ng/g of recombinant DNA.

Embodiment 131. The method of any of the preceding embodiments, wherein the microbial fermentation product comprises less than 1 ng/g of recombinant DNA.

Embodiment 132. The method of any of the preceding embodiments, wherein the microbial fermentation product comprises less than 0.5 ng/g of recombinant DNA.

Embodiment 133. The method of any of the preceding embodiments, wherein the microbial fermentation product comprises less than 0.1 ng/g of recombinant DNA.

Embodiment 134. The method of any of the preceding embodiments, wherein the microbial fermentation product comprises less than 0.05 ng/g of recombinant DNA.

Embodiment 135. The method of any of the preceding embodiments, wherein the microbial fermentation product comprises less than 0.01 ng/g of recombinant DNA.

Embodiment 136. The method of any of the preceding embodiments, wherein the microbial fermentation product comprises the DNase.

Embodiment 137. A microbial fermentation product comprising less than 10 ng/g of recombinant DNA.

Embodiment 138. The microbial fermentation product of the preceding embodiment, which comprises less than 5 ng/g of recombinant DNA.

Embodiment 139. The microbial fermentation product of any of the preceding embodiments, which comprises less than 1 ng/g of recombinant DNA.

Embodiment 140. The microbial fermentation product of any of the preceding embodiments, which comprises less than 0.5 ng/g of recombinant DNA.

Embodiment 141. The microbial fermentation product of any of the preceding embodiments, which comprises less than 0.1 ng/g of recombinant DNA.

Embodiment 142. The microbial fermentation product of any of the preceding embodiments, which comprises less than 0.05 ng/g of recombinant DNA.

Embodiment 143. The microbial fermentation product of any of the preceding embodiments, which comprises less than 0.01 ng/g of recombinant DNA.

Embodiment 144. The microbial fermentation product of any of the preceding embodiments, which further comprises a fungal DNase or a variant thereof.

Embodiment 145. The microbial fermentation product of the preceding embodiment, wherein the fungal DNase or variant thereof is a NUC3 nuclease.

Embodiment 146. The microbial fermentation product of any of the preceding embodiments, which comprises a protein of interest.

Embodiment 147. The microbial fermentation product of the preceding embodiment, wherein the protein of interest is a recombinant protein of interest.

Embodiment 148. The microbial fermentation product of any of the preceding embodiments, which comprises at least 0.1% w/w of a protein of interest.

Embodiment 149. The microbial fermentation product of any of the preceding embodiments, which comprises at least 0.5% w/w of a protein of interest.

Embodiment 150. The microbial fermentation product of any of the preceding embodiments, which comprises at least 1% w/w of a protein of interest.

Embodiment 151. The microbial fermentation product of the preceding embodiment, wherein the protein of interest is an enzyme.

Embodiment 152. The microbial fermentation product of any of the preceding embodiments, which is produced by the method of any of the preceding embodiments.

### EXAMPLES

Chemicals were commercial products of at least reagent grade.

### DNases

“Bacterial nuclease” is the Bacillus cibi DNase having the amino acid sequence shown as SEQ ID NO: 1.

“Fungal nuclease” is the Aspergillus oryzae DNase having the amino acid sequence shown as SEQ ID NO: 2.

Other DNases were also used for comparison. The donor strains are indicated in the corresponding examples.

Detection of gDNA and resDNA have been conducted by PCR and digital PCR (dPCR) analysis described in Example 1. The specific analysis used will be stated in the individual examples.

### Example 1

### Detection of Residual Recombinant DNA

The method used for demonstration of the absence of residual recombinant DNA in each sample can be divided into 2 steps: step i) extraction of DNA from the samples, including a lysis step, and step ii) detection of the recombinant DNA (target) by PCR amplification using either traditional PCR technique followed by gel electrophoresis or digital PCR technique.

### Extraction of DNA Including Lysis Step

The DNA extraction method is based on a commercial kit (Maxwell RSC PureFood GMO & Authentication kit, Promega). The standard treatment with Proteinase K was modified to enable efficient removal of proteins and thereby decrease the probability of PCR inhibition by the sample matrix.

At first, 200 μl of sample was treated with Proteinase K (QIAgen cat. no. 19133) in CTAB buffer at a final concentration of 12.5 mg Proteinase K per ml sample. The mixture was then incubated at 40° C. for 1 hour to ensure efficient removal of proteins. Subsequently, the DNA was extracted using the Maxwell RSC instrument by following the procedure described in the commercial kit, however, the DNA was eluted in 55 μl of DNase-free water.

### Detection of Residual Recombinant DNA Using PCR Amplification

PCR amplification is a common method used for the detection of very small amounts of target DNA. In these experiments, both traditional PCR and digital PCR techniques were used. The difference between the two PCR methods is as follows: traditional PCR technique is a qualitative method where the amplified PCR target is detected by agarose gel electrophoresis. Whereas digital PCR technique is a quantitative method based on the TaqMan technology where the amount of amplified PCR target can be quantified.

The primes used for both techniques are unique for the recombinant DNA in question (Table 1) and will amplify target fragments of a size less than 150 bp. For the digital PCR technique, which is based on the TaqMan technology, a sequence-specific oligonucleotide (Probe) with a fluorophore and a quencher moiety attached is also needed (Table 1). The design of primer, probe and amplicon was based on basic considerations using webtools as described by Rodriguez et al in Chapter 3: Design of primers and Probes for Quantitative Real-time PCR methods in Methods in Molecular Biology 1275, Springer Protocols (editor C. Basu)

TABLE 1
PCR primers used for traditional
PCR amplification.

| Forward primer | ID | ADO3 |
|---|---|---|
| | Seq. | 5′-TACGCGGTTAAAGTCAATG-3′ |
| | | (SEQ ID NO: 5) |
| Reverse primer | ID | ADO24 |
| | Seq. | 5′-TTCGCAGGATAGCCAATTGTA-3′ |
| | | (SEQ ID NO: 11) |
| Target size | (bp) | 104 |

TABLE 2
PCR primers and Probe used for the
digital PCR amplification.

| Forward primer | ID | ADO5 |
|---|---|---|
| | Seq. | 5′-GAATAAAGAGGAGGATCCG-3′ |
| | | (SEQ ID NO: 7) |
| Reverse primer | ID | ADO28 |
| | Seq. | 5′-CGACGATCATGGAACGGATT-3′ |
| | | (SEQ ID NO: 9) |
| Probe | ID | GEMA010 |
| | Seq. | 56-FAM/TCTTCATTCTGATGAAGGTTGT |
| | | TC/BHQ_1 |
| | | (SEQ ID NO: 10) |
| Target size | (bp) | 140 |

TABLE 3
PCR primers used for the traditional
PCR amplification.

| Forward primer | ID | ADO3 |
|---|---|---|
| | Seq. | 5′-TACGCGGTTAAAGTCAATG-3′ |
| | | (SEQ ID NO: 5) |
| Reverse primer | ID | ADO4 |
| | Seq. | 5′-ACAAAGCATTCTGAAGCTGG-3′ |
| | | (SEQ ID NO: 6) |
| Target size | (bp) | 1096 |

TABLE 4
PCR primers used for the traditional
PCR amplification.

| Forward primer | ID | ADO5 |
|---|---|---|
| | Seq. | 5′-GAATAAAGAGGAGGATCCG-3′ |
| | | (SEQ ID NO: 7) |
| Reverse primer | ID | ADO6 |
| | Seq. | 5′-GGGCGGAGCAGACCTCGAAC-3′ |
| | | (SEQ ID NO: 8) |
| Target size | (bp) | 1076 |

TABLE 5
PCR primers and Probe used for the
digital PCR amplification.

| Forward primer | ID | ADO3 |
|---|---|---|
| | Seq. | 5′-TACGCGGTTAAAGTCAATG-3′ |
| | | (SEQ ID NO: 5) |
| Reverse primer | ID | ADO24 |
| | Seq. | 5′-TTCGCAGGATAGCCAATTGTA-3′ |
| | | (SEQ ID NO: 11) |
| Probe | ID | GEMA12 |
| | Seq. | 56-FAM/ ACAGCGGATOTTCAGGAAACAC |
| | | GA/BHQ_1 |
| | | (SEQ ID NO: 12) |
| Target size | (bp) | 104 |

TABLE 6
PCR primers and Probe used for the
digital PCR amplification.

| Forward primer | ID | ADO73 |
|---|---|---|
| | Seq. | 5′-CCTGGAATGCTGTTGGATTGTA-3′ |
| | | (SEQ ID NO: 13) |
| Reverse primer | ID | ADO74 |
| | Seq. | 5′-CTGTCTCATGGCAGCATCTT-3′ |
| | | (SEQ ID NO: 14) |
| Probe | ID | GEMA15 |
| | Seq. | 56-FAM/ TGATTTCTCTCTCAGCTGAAAC |
| | | GA/BHQ_1 |
| | | (SEQ ID NO: 15) |
| Target size | (bp) | 118 |

### Traditional PCR Method

The PCR amplification was performed on 10 μL of extracted DNA using the relevant primer set for the given target at a concentration of 400 nM for each primer. Per reaction, one tube of Illustra PureFood PCR beads system from GF Healthcare (cat. No. 27-9557-02) was used. The PCR reactions were running under the following thermocycler conditions:

| Temperature | Time | |
|---|---|---|
| (° C.) | (seconds) | Cycles |
| 96 | 300 | 1 |
| 94 | 10 | 10 |
| 65 | 30 | |
| -1° C./cycle | | |
| 72 | 60 | |
| 94 | 10 | 25 |
| 55 | 30 | |
| 72 | 60 | |
| 72 | 420 | 1 |
| 4 | Forever | — |

Following PCR amplification, the PCR reactions were visualized on a 2.2% agarose gel (FlashGel system from Lonza, cat. no. 57031). FlashGel DNA marker 100 bp-4 kb (Lonza cat. no. 50473) was used for band size estimation.

### Digital PCR Method

Digital PCR (dPCR) uses the procedure of end-points PCR but splits the PCR reaction into many single partitions, in which the template is randomly distributed across all available partitions. After PCR, the amplification is detected by measuring the fluorescent in all positive partitions. Using Poisson statistics, the average amount of target DNA per sample can be calculated. The number of filled partitions is identified by a reference fluorescent dye present in the reaction mixture. Absolute quantification of the amount of target DNA in each sample can then be calculated.

For these experiments, the digital system QIAcuity ONE from QIAGEN was used together with the QIAcuity Probe PCR kit (Cat. no. 250101). The kit contains a 4× concentrated, ready-to-use Master mix optimized for use with the QIAcuity nanoplates (cat. no. 25001) which distributes each sample into 26,000 partitions. The procedure described by the supplier has been optimized with regard to Primer and Probe concentrations as well as for the PCR cycling conditions. For these experiments, a primer concentration of 800 nM of each and a probe concentration of 400 nM were used.

Extracted DNA should be fragmented by restriction digestions before partitioning to ensure even distribution of templates throughout the QIAcuity Nanoplate. For these experiments, the restriction enzyme EcoRI was chosen at a concentration of 0.25 U per 40 μl reaction. This restriction enzyme was chosen since it does not cut within the target fragment. For each dPCR reaction, 5 μl of template DNA was used in a total reaction volume of 40 μl which was obtain using DNase-free water.

The mixture was then transferred to the nanoplate, sealed, and incubated at room temperature for a minimum of 10 minutes for the restriction enzyme to work.

Immediately, thereafter, the nanoplate was run at the following thermal cycling conditions:

| Temperature | Time | |
|---|---|---|
| (° C.) | (seconds) | Cycles |
| 95 | 120 | 1 |
| 95 | 15 | |
| 60 | 30 | 40 |
| 72 | 30 | |

The result of the dPCR analysis is given as copies of target PCR fragment per μl of total reaction volume. Based on this value, the amount of ng recombinant DNA per g sample in the starting material can be calculated based on the following assumptions:
100% effective DNA purification obtained.
Average weight of Bacillus genome is estimated to be 4.2×10⁶ base pair (ref. J.T. Trevors, 1996, Genome size in bacteria. Antonie van Leeuwenhocek 69 (4): 293-303).
The molecular weight of a base pair is 650 Daltons.
One Dalton corresponds to 1.6605×10⁻¹⁵ ng.
The weight of Bacillus genome is estimated to be 4.5×10⁻⁶ ng.

The content of residual recombinant DNA in the original sample can then be calculated using the following equation:

$$ng DNA per ml sample = 0.04 \times Df \times D$$

where Df is the dilution factor of the DNA preparation prior to dPCR analysis and D is the results of the dPCR analysis expressed as copies per μl in a total reaction volume of 40 μl.

### Example 2

Degradation of Genomic DNA in Distilled Water with a Bacterial Nuclease from Bacillus Cibi and a Fungal Nuclease from Aspergillus oryzae

### Experimental Procedure

To understand nuclease degradation of purified genomic Bacillus licheniformis DNA (gDNA) in a simple ‘clean’ matrix, all samples in Example 2 were prepared in distilled water (DW). To evaluate gDNA degradation, two positive control samples were prepared in DW with a total volume of 300 μL per sample. The control samples were spiked with 1000 ng/g gDNA at pH 9. A total of 5 mM MgCl₂ was added to one of the two control samples. Another positive control sample was prepared in DW and spiked with 10 ng/g gDNA. gDNA degradation using a bacterial nuclease was investigated with a dose-response, ending with final concentrations in the range of 0.0005 g/L to 0.5 g/L in factor 10 concentration increments. In addition, to an extra sample with a final concentration of 0.005 g/L, MgCl₂ (co-factor) was added to a final concentration of 5 mM. A final concentration of 0.5 g/L was used for the fungal nuclease. The total reaction volume was 300 UL at pH 9 and the samples were incubated at 37° C. for 24 hours with gentle agitation. The nuclease reactions were quenched by freezing the samples after the incubation time. The ability of the nuclease to degrade gDNA was evaluated by traditional PCR analysis using the primers shown in Table 4.

### Results

Both the bacterial and fungal nucleases were able to degrade gDNA in DW to <10 ng/g at any tested concentration and independent of if additional MgCl₂ was added or not (Table 7).

TABLE 7

| Sample type | Concentration of gDNA | |
|---|---|---|
| Control samples | 10 and 1000 | ng/g |
| Fungal nuclease in a 1000 ng/g sample | <10 | ng/g |
| (0.5 g/L of nuclease) | | |
| Bacterial nuclease in a 1000 ng/g sample | <10 | ng/g |

(0.0005-0.5 g/L of nuclease)

### Example 3

Degradation of DNA in an Enzyme Concentrate with a Bacterial and a Fungal Nuclease

### Experimental Procedure

All samples in Example 3 were prepared using an enzyme concentrate recovered from a Bacillus licheniformis fermentation broth (FB). The enzyme concentrate had a residual DNA (resDNA) concentration of >>10 ng/g. Residual DNA is the amount of recombinant DNA derived from the B. licheniformis host cells. ResDNA degradation using the bacterial nuclease was investigated with a dose-response, ending with final concentrations in the range of 0.0005 g/L to 0.5 g/L in factor 10 concentration increments. In addition, to an extra sample with a final concentration of 0.005 g/L, MgCl₂ (co-factor) was added to a final concentration of 5 mM. A final concentration of 0.5 g/L was used for the fungal nuclease. The total reaction volume was 400 μL at pH 6.5 and the samples were incubated at 37° C. for 24 hours with gentle agitation. The nuclease reactions were quenched by freezing the samples after the incubation time. Two positive control samples were used: the enzyme concentrate and DW spiked with 10 ng/g gDNA. The resDNA degradation was evaluated by traditional PCR analysis using the primers shown in Table 3.

### Results

The fungal nuclease was able to degrade bacterial host cell resDNA in the enzyme concentrate to <<10 ng/g. The bacterial nuclease was not able to degrade any significant level of bacterial host cell resDNA at any tested concentration and independent of if additional MgCl₂ was added or not (Table 8).

TABLE 8

| Sample type | Concentration of resDNA | |
|---|---|---|
| Control sample - enzyme concentrate | >>10 | ng/g |
| Control sample | 10 | ng/g |
| Fungal nuclease in an enzyme concentrate | <<10 | ng/g |
| (0.5 g/L of nuclease) | | |
| Bacterial nuclease in an enzyme concentrate | ≥10 | ng/g |

(0.0005-0.5 g/L of nuclease)

### Example 4

Degradation of DNA in an Enzyme Concentrate with the Bacterial and Fungal Nuclease at Different pHs

### Experimental Procedure

Since the fungal nuclease was the only nuclease that was efficient in degrading resDNA in Example 3, the trial was repeated in an enzyme concentrate recovered from a Bacillus licheniformis FB at different pHs. The enzyme concentrate had a resDNA concentration of >10 ng/g. A final concentration of 0.5 g/L was used for both nucleases. The total reaction volume was 400 μL at pH 4, 5, 6, 7 and 8 for the fungal nuclease and pH 4, 6 and 8 for the bacterial nuclease and the samples were incubated at 37° C. for 24 hours with gentle agitation. The nuclease reactions were quenched by freezing the samples after the incubation time. The enzyme concentrate without any addition of nuclease was used as a positive control sample. The resDNA degradation was evaluated by traditional PCR analysis using the primers shown in Table 1.

### Results

The fungal nuclease was able to degrade bacterial host cell resDNA in the enzyme concentrate to <<10 ng/g at all pHs investigated but was most efficient at pH 4 (<<<10 ng/g). The bacterial nuclease was not able to degrade any significant level of bacterial host cell resDNA in the enzyme concentrate at any of the tested pHs but a minor effect was observed at pH 4 (Table 9).

TABLE 9

| Sample type | Concentration of resDNA | |
|---|---|---|
| Control sample - enzyme concentrate | >10 | ng/g |
| Fungal nuclease - pH 4 | <<<10 | ng/g |
| Fungal nuclease - pH 5 | <<10 | ng/g |
| Fungal nuclease - pH 6 | <<10 | ng/g |
| Fungal nuclease - pH 7 | <<10 | ng/g |
| Fungal nuclease - pH 8 | <<10 | ng/g |
| Bacterial nuclease - pH 4 | <10 | ng/g |
| Bacterial nuclease - pH 6 | ≥10 | ng/g |
| Bacterial nuclease - pH 8 | ≥10 | ng/g |

### Example 5

Degradation of DNA in Fermentation Broth and Flocculated Fermentation Broth with the Fungal Nuclease at Different pHs

### Experimental Section

All samples in Example 5 were prepared using a FB or flocculated fermentation broth (fFB) from Bacillus licheniformis.

### Preparation of the Fermentation Broth Samples

The fungal nuclease was added directly to the FB at pH 7.5+/−0.5 to achieve a final nuclease concentration of 0.5 g/L. The FB without any addition of fungal nuclease was used as a positive control sample. The samples were incubated at 37° C. for 24 hours with gentle agitation. The nuclease reaction was quenched by freezing the sample after the incubation time. The resDNA degradation was evaluated by dPCR analysis using the primers/probe shown in Table 2.

### Preparation of the fFB Samples

To produce a fFB, the FB was treated as follows:
1. Dilution with water
2. Addition of a divalent salt
3. Addition of a poly aluminum chloride (PAC)
4. pH adjustment to 4, 6 or 8
5. Addition of the fungal nuclease to achieve a final concentration of 0.5 g/L
6. Incubation at 37° C. for 24 hours with gentle agitation

The positive control samples were produced in the same way but without step 5. The nuclease reaction was quenched by freezing the samples after the incubation time. The resDNA degradation was evaluated by dPCR analysis using the primers/probe shown in Table 2.

### Results

The fungal nuclease significantly degraded the bacterial host cell resDNA comparing to the control samples independently if it was added to the FB or the fFB. In addition, the nuclease efficiency was similar at all tested pHs (Table 10).

TABLE 10

| | Concentration of resDNA | Concentration of |
|---|---|---|
| | without the fungal nuclease | resDNA with the |
| Sample type | (control samples) | fungal nuclease |
| FB | >3500 ng/g | <2 ng/g |
| fFB at pH 4 | >3500 ng/g | <0.005 ng/g |
| fFB at pH 6 | >1000 ng/g | <0.005 ng/g |
| fFB at pH 8 | >500 ng/g | <0.005 ng/g |

### Example 6

Degradation of DNA During Recovery of an Enzyme from a Fermentation Broth

### Experimental Section

All samples in Example 6 are from different streams during recovery of an enzyme from a Bacillus licheniformis FB.

### Preparation of the FB

The fungal nuclease was added in the tank before the fermentation started to achieve a final nuclease concentration of 0.5 g/L+/−0.1. The pH during the fermentation and recovery was 7.5+/−0.5. A FB without any addition of fungal nuclease was used as a positive control batch. The FB was harvested at and incubated at 5° C. for 22-24 hours before recovery started. The nuclease reaction was quenched by freezing the sample after the incubation time. The resDNA degradation was evaluated by dPCR analysis using the primers/probe shown in Table 2.

### The Recovery Process

To produce a fFB, the FB was treated as follows:
1. Dilution with water
2. Addition of a divalent salt
3. Addition of a poly aluminum chloride (PAC)
4. pH adjustment to 7.5+/−0.5
5. Addition of a cationic polymer
6. Addition of an anionic polymer
7. The fFB was centrifuged to separate the liquid phase (supernatant) from the biomass
8. The supernatant was filtered on filters with a cut-off size of 5.0-0.3 μm to produce a filtrate
9. The filtrate was concentrated using ultra filtration (UF) with a membrane cut-off size of 10 kDa to produce a UF-concentrate

### Results

The fungal nuclease significantly degraded the bacterial host cell resDNA in the FB, supernatant of the fFB and in the UF-concentrate comparing to the control FB (Table 11).

TABLE 11

| | Concentration of resDNA | |
|---|---|---|
| | without the fungal nuclease | Concentration of resDNA |
| Sample type | (control samples) | with the fungal nuclease |
| FB | >>>1000 ng/g | >1000 ng/g |
| Supernatant of fFB | >900 ng/g | <150 ng/g |
| UF-concentrate | >>10 ng/g | <10 ng/g |

### Example 7

Degradation of DNA in an Enzyme Concentrate with DNases from Different Organisms

### Experimental Procedure

All samples in Example 7 were prepared using an enzyme concentrate recovered from a B. licheniformis FB which had a resDNA concentration of >>10 ng/g. ResDNA degradation using nucleases from different organisms was investigated with final nuclease concentrations of 0.5 g/L. The enzyme concentrate had a pH of 5.9 and the samples were incubated at 25° C. for 1 hour with gentle agitation. The nuclease reactions were quenched by freezing the samples after the incubation time. The enzyme concentrate was used as positive control sample. The resDNA degradation was evaluated by dPCR analysis using the primers/probe shown in Table 5.

### Results

The fungal nuclease (from A. oryzae) degraded the bacterial host cell resDNA <<1 ng/g in the enzyme concentrate, where none of the other tested nuclease from different organisms were able to degrade the resDNA <10 ng/g (Table 12).

TABLE 12

| Sample type | Concentration of resDNA |
|---|---|
| Control sample - enzyme concentrate | >>10 ng/g |
| Fungal nuclease (from A. oryzae) | <<1 ng/g |
| Bacterial nuclease (from B. cibi) | >>10 ng/g |
| DNase from Neosartorya massa | >>10 ng/g |
| DNase from Arthrographis sp. 07MA20 | >>10 ng/g |
| DNase from Rhizoctonia solani | >>10 ng/g |
| DNase from Morchella costata | >>10 ng/g |
| DNase from Pyrenochaetopsis sp. | >>10 ng/g |
| DNase from Arthrinium arundinis | >>10 ng/g |
| DNase from Cladosporium cladosporioides | >>10 ng/g |
| DNase from Penicillium quercetorum | >>10 ng/g |
| DNase from Phialophora geniculata | >>10 ng/g |
| DNase from Acremonium chrysogenum | >>10 ng/g |

### Example 8

Degradation of DNA in an Enzyme Concentrate with DNases from Different Organisms

### Experimental Procedure

All samples in Example 8 were prepared using an enzyme concentrate recovered from a Bacillus licheniformis FB which had a resDNA concentration of >10 ng/g. ResDNA degradation using nucleases from different organisms was investigated with final nuclease concentrations of 0.5 g/L. The enzyme concentrate had a pH of 5.0 and the samples were incubated at 25° C. for 1 hour with gentle agitation. The nuclease reactions were quenched by freezing the samples after the incubation time. The enzyme concentrate was used as positive control sample. The resDNA degradation was evaluated by dPCR analysis using the primers/probe shown in Table 2.

### Results

The fungal nuclease (from A. oryzae) degraded the bacterial host cell resDNA <<<1 ng/g in the enzyme concentrate. The bacterial nuclease and nucleases from Neosartorya massa, Arthrographis sp. 07MA20, Rhizoctonia solani and Morchella costata degraded resDNA <10 ng/g. The nucleases from Pyrenochaetopsis sp., Arthrinium arundinis, Cladosporium cladosporioides, Penicillium quercetorum, Phialophora geniculate and Acremonium chrysogenum were not able to degrade resDNA <10 ng/g (Table 13).

TABLE 13

| Sample type | Concentration of resDNA |
|---|---|
| Control sample - enzyme concentrate | >10 ng/g |
| Fungal nuclease (from A. oryzae) | <<<1 ng/g |
| Bacterial nuclease (from B. cibi) | <10 ng/g |
| DNase from Neosartorya massa | <10 ng/g |
| DNase from Arthrographis sp. 07MA20 | <10 ng/g |
| DNase from Rhizoctonia solani | <10 ng/g |
| DNase from Morchella costata | <10 ng/g |
| DNase from Pyrenochaetopsis sp. | >10 ng/g |
| DNase from Arthrinium arundinis | >10 ng/g |
| DNase from Cladosporium cladosporioides | >10 ng/g |
| DNase from Penicillium quercetorum | >10 ng/g |
| DNase from Phialophora geniculata | >10 ng/g |
| DNase from Acremonium chrysogenum | >10 ng/g |

### Example 9

Degradation of DNA in an Enzyme Concentrate with DNases from Different Organisms

### Experimental Procedure

All samples in Example 9 were prepared using an enzyme concentrate recovered from a Bacillus subtilis FB which had a resDNA concentration of >>>10 ng/g. ResDNA degradation using nucleases from different organisms was investigated with final nuclease concentrations of 0.5 g/L. The enzyme concentrate had a pH of 6.2 and the samples were incubated at 25° C. for 1 hour with gentle agitation. The nuclease reactions were quenched by freezing the samples after the incubation time. The enzyme concentrate was used as positive control sample. The resDNA degradation was evaluated by dPCR analysis using the primers/probe shown in Table 6.

### Results

The fungal nuclease (from A. oryzae) degraded the bacterial host cell resDNA <<1 ng/g in the enzyme concentrate. The bacterial nuclease and nucleases from Neosartorya massa, Pyrenochaetopsis sp., Arthrinium arundinis and Phialophora geniculata were not able to degrade resDNA <10 ng/g (Table 14).

TABLE 14

| Sample type | Concentration of resDNA |
|---|---|
| Control sample - enzyme concentrate | >>>10 ng/g |
| Fungal nuclease (from A. oryzae) | <<1 ng/g |
| Bacterial nuclease (from B. cibi) | >>10 ng/g |
| DNase from Neosartorya massa | >>10 ng/g |
| DNase from Pyrenochaetopsis sp. | >>10 ng/g |
| DNase from Arthrinium arundinis | >>10 ng/g |
| DNase from Phialophora geniculata | >>10 ng/g |

1. A method for reducing the amount of DNA in a microbial fermentation product, said method comprising
(a) providing a fermentation broth comprising microbial host cells, recombinant DNA from the microbial host cells, and a protein of interest produced by the microbial host cells;
(b) subjecting the fermentation broth to a flocculation or precipitation step to provide a fermentation broth supernatant; and
(c) subjecting the fermentation broth supernatant to a membrane filtration step to provide a fermentation product, where the membrane has a size exclusion limit of less than 100 kDa or less than 1 μm;
wherein a fungal DNase or a variant thereof is added to the fermentation medium before or during the fermentation, to the fermentation broth in or after step (a), to the fermentation broth supernatant after step (b), or to the fermentation product after step (c).

2. The method of the preceding claim, wherein the protein of interest is heterologous to the microbial host cells.

3. The method of claim 1, wherein the fermentation broth in step (a) comprises the protein of interest in an amount of at least 0.1% w/w.

4. The method of claim 1, wherein the protein of interest is an enzyme, heme-containing protein, cell signaling protein, or ligand binding protein.

5. The method of claim 1, wherein the membrane filtration in step (c) comprises a microfiltration step.

6. The method of claim 1, wherein the membrane filtration in step (c) comprises an ultra-filtration step.

7. The method of claim 1, wherein some or all of the microbial host cells in step (a) have been disrupted/homogenized mechanically or enzymatically.

8. The method of claim 1, wherein the fungal DNase or variant thereof has at least 60% amino acid sequence identity to SEQ ID NO: 2, and where the variant exhibits DNase activity.

9. The method of claim 1, wherein the fungal DNase or variant thereof comprises the DNase_NucA_NucB domain, and further comprises the motif [LV][PTA][FY][DE][VAGPH]D[CFY][WY][AT][IM]L[CYQ] and/or any of the motifs GPYCK (SEQ ID NO: 3) or WF[QE]IT (SEQ ID NO: 4).

10. The method of claim 1, wherein the microbial host cell is a strain selected from the group consisting of Candida, Hansenula, Kluyveromyces, Pichia, Saccharomyces, Schizosaccharomyces, Yarrowia, Acremonium, Aspergillus, Fusarium, Humicola, Mucor, Myceliophthora, Neurospora, Penicillium, Thielavia, Tolypocladium, and Trichoderma.

11. The method of claim 1, wherein the microbial host cell is a bacterial host cell.

12. The method of claim 1, wherein the microbial fermentation product comprises less than 10 ng/g of recombinant DNA.

13. A microbial fermentation product comprising less than 10 ng/g of recombinant DNA.

14. The microbial fermentation product of claim 13, which further comprises a fungal DNase or a variant thereof.

15. The microbial fermentation product of claim 13, which further comprises a protein of interest.

16. The microbial fermentation product of claim 15, wherein the protein of interest is a recombinant protein of interest.

17. The microbial fermentation product of claim 13, which is produced by the method of any of the preceding claims.
